(19) United States
(12) Patent Application Publication
(10) Pub. No.: US 2005/0143582 A1
(43) Pub. Date: Jun. 30, 2005

(54) Method for producing substituted 3-phenylamino-propane-1,2-diols

(76) Inventors: Ekkehard Bartmann, Erzhausen (DE); Ingo Sandner, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/504,063

(22) PCT Filed: Jan. 14, 2003
(86) PCT No.: PCT/EP03/00258

(30) Foreign Application Priority Data
Feb. 14, 2002 (DE) 102-06-012.6

(51) Int. Cl. C07D 263/16

(52) U.S. Cl. 548/229; 558/418

(57) ABSTRACT

The present invention relates to an improved process for the preparation of 3-phenylaminopropane-1,2-diols. 3-Phenylaminopropane-1,2-diols are valuable intermediates which can be converted further into 3-phenyloxazolidinone compounds.

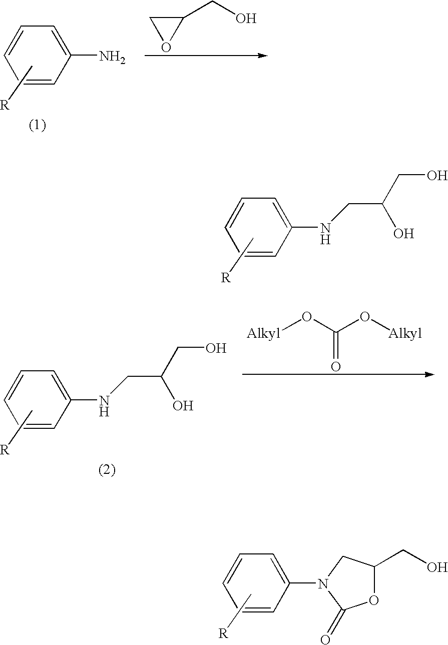

The present invention relates to an improved process for the preparation of 3-phenylaminopropane-1,2-diols. 3-Phenylaminopropane-1,2-diols are valuable intermediates which can, for example, be converted further into phenyloxazolidinone compounds.

Phenyloxazolidinone compounds, in particular 4-amidinophenyloxazolidinone compounds, are disclosed, for example, in WO 01/40201 A1, EP 0 623 615 A1 and EP 0 741 133 A1. The compounds described therein inhibit, in particular, the binding of fibrinogen, fibronectin and of von Wille-brand factor to the fibrinogen receptor of the blood platelets (glycoprotein IIb/IIIa) and also the binding thereof and of further adhesive proteins, such as vitronectin, collagen and laminin, to the corresponding receptors on the surface of various types of cell. By blocking the glycoprotein IIb/IIIa receptors, they inhibit metastasis, i.e. the spread of tumour cells, and can therefore be employed, for example, in tumour treatment.

As described, for example, in EP 0 623 615 A1, the pharmacologically active compounds are prepared by firstly reacting an appropriately substituted aniline with glycidol to give 3-phenylaminopropane-1,2-diol, and subsequently reacting this with a carbonic acid derivative to give the oxazolidinone compound. The oxalzolidinone compound obtained can then be converted in further steps into the particular pharmacologically active oxalzolidinone derivative.

The reaction is depicted below:

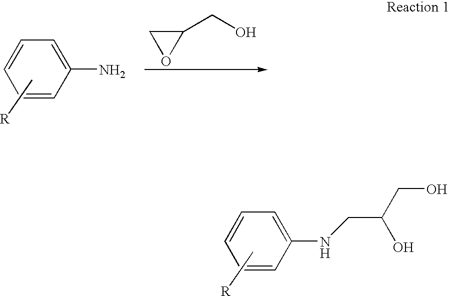

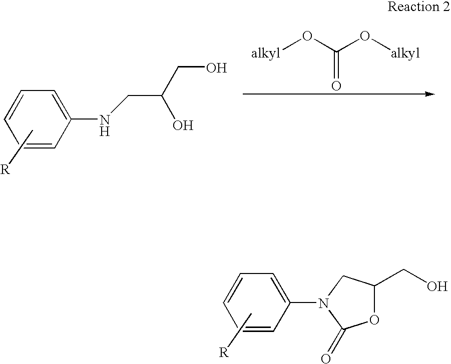

The reaction of the substituted aniline with glycidol (reaction 1) proceeds very slowly or not at all in organic polar or nonpolar solvents. It has therefore is hitherto been carried out in water as solvent. However, it does not proceed satisfactorily in water either. Besides the desired reaction, further side-reactions occur, and consequently glycidol has to be employed in a virtually two-fold excess in relation to the substituted aniline. In spite of this excess, the yields, even after optimisation of the reaction conditions, such as temperature and reaction time, are at best about 65% based on the substituted aminobenzonitrile and even only about 35% based on glycidol. Thus, approximately two thirds of the glycidol are lost in the reaction. This is unsatisfactory if only because glycidol is expensive, in particular if chiral glycidol is employed.

In the synthesis of many pharmacologically interesting oxazolidinone compounds, the reaction of substituted aniline with glycidol to give a 3-phenylaminopropane-1,2-diol (reaction 1) is the first step of a synthesis which proceeds over a large number of steps. For example, in the preparation of GPIIb/IIIa antagonists, such as gantofiban, by reaction of the aniline with glycidol, nine further synthetic steps are necessary (Bioorganic & Medicinal Chemistry Letters, Vol 6, No 20, 2425-2430, (1996)). The high preparation costs associated with the unsatisfactory reaction of the aniline derivative with glycidol are thus translated multiplied a number of times into the preparation costs for the respective end product. In addition, the product impurities arising from the unsatisfactory reaction have to be removed in further additional steps. In addition to further increased costs, this also means significantly greater consumption of time.

Many attempts to improve the reaction have not produced the desired result. Thus, the addition of possible catalysts (protonic and Lewis acids, bases, salts of oxophilic metal cations, transition-metal compounds) gave at most the same yields as without addition, but usually gave worse results. The product was often highly impure. Even on addition of catalysts which, according to literature data, effect significant improvements in reactions of other epoxides, no improvement was obtained. Catalysts described as suitable are LiClO₄, LiBF₄, Mg(ClO₄)₂, NaClO₄, CaCl₂, ZnCl₂, Zn(OSO₂CF₃)₂ (M. Chini, P. Crotti, F. Macchia; Tetrahedron Letters 31 (1990) 4661) and Li(OSO₂CF₃)₂, Sc(OSO₂CF₃)₂ (J. Augé, F. Leroy; Tetrahedron Letters 37 (1996) 7715).

The object of the present invention was to provide an improved synthesis process which avoids the above-described disadvantages of the previous process. In particular, the aim is to suppress side-reactions and to increase the yield.

Surprisingly, it has been found that the yield of the process and thus the completeness of the conversion into the desired product is considerably improved if the substituted aniline is employed in the reaction as solvent for the glycidol. The invention therefore relates to a process for the preparation of substituted 3-phenylaminopropane-1,2-diols by reaction of an appropriately substituted aniline with glycidol, which is characterised in that glycidol dissolved in the melt of the substituted aniline is reacted.

The reaction proceeds at least as quickly in molten substituted aniline as in water. This is amazing, if only because organic solvents otherwise, as described above, result in a significant impairment of the reaction.

Mono-, di-, tri- or tetrasubstituted aniline can be employed in the process according to the invention, with preference being given to monosubstituted aniline. In monosubstituted aniline, the substituent may be present in the 4-, 3- or 2-position. Aniline is preferably in the 3- or 4-position, particular preference being given to substitution in the 4-position. Substituents which may be present are, in particular, CN, CO₂A, CO(NRQ), where R and Q=H or A, and Hal. The substituent is particularly preferably the nitrile group. Particular preference is given to aminobenzonitrile, in particular4-aminobenzonitrile. These can the be converted in further steps into pharmacologically valuable 4-amidinophenyloxazolidinone compounds.

In the above formulae, A is alkyl, is linear or branched, and has from 1 to 8, preferably 1, 2, 3, 4, 5 or 6, carbon atoms. A is preferably methyl, furthermore ethyl, n-propyl, isopropyl, n-butyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, heptyl or octyl. Embodiments of A which are furthermore preferred are the said alkyl groups, but which may be monosubstituted or polysubstituted by Hal or NO₂, preferably trifluoromethyl, 2,2,2-trifluoroethyl or 2-nitroethyl, or alkyl groups whose carbon chain may be interrupted by —O—, preferably —CH₂—O—CH₃, —CH₂—O—CH₂—CH₃ or —CH₂—CH₂-O—CH₃.

A is particularly preferably methyl or ethyl. Hal is preferably F, Cl, Br or iodine. Hal is particularly preferably F or Cl.

Depending on the desired product, glycidol can be employed in the process according to the invention in the form of the racemate or in the form of one of its optically active enantiomers R-(+)-glycidol or S-(−)-glycidol. Glycidol is preferably employed in the form of one of its optically active enantiomers, particularly preferably R-(+)-glycidol.

The reaction of the substituted aniline is described below using the example of aminobenzonitrile. The reaction with other substituted anilines is carried out analogously in the melt of the respective substituted aniline.

Aminobenzonitrile melts in pure form at 85° C. However, addition of a few percent of another substance advantageously enables the melting point to be lowered to about 40° C. Glycidol itself is advantageously employed for this purpose.

A particularly suitable procedure for carrying out the reaction is firstly to introduce the aminobenzonitrile, to warm the latter to from about 40 to 45° C., and subsequently to add from 5 to 10 mol % of glycidol, based on one mole of initially introduced aminobenzonitrile. When melting is complete, the mixture is warmed to from about 60 to 70° C., and the desired residual amount of glycidol is slowly added dropwise (over the course of from about 1 to 3 hours). On addition of less than half a mole of glycidol, based on one mole of aminobenzonitrile, the mixture is stirred for a further 1 to 2 hours; on addition of more than half a mole of glycidol, based on one mole of aminobenzonitrile, the mixture is stirred at elevated temperature for about a further 3 to 10 hours.

The yields are excellent compared with the previous process; on use of less than half a mole of glycidol, based on one mole of aminobenzonitrile, up to 75% of the desired product, based on the amount of glycidol, can be achieved. If the amount of glycidol is increased, the yield relative to the glycidol drops again, but naturally increases in relation to the aminobenzonitrile.

Particularly high yields are obtained if the substituted aniline and glycidol are reacted with one another in a molar ratio of from 1.5 to 2.5:1, preferably in a molar ratio of approximately 2:1. An advantageous embodiment of the process according to the invention is thus characterised in that the substituted aniline and glycidol are reacted with one another in a molar ratio of from 1.5 to 2.5:1, preferably in a molar ratio of approximately 2:1.

According to an advantageous embodiment of the process according to the invention, excess unreacted substituted aniline serving only as solvent in the reaction and remaining in the resultant reaction product is subsequently removed by treatment with a suitable solvent, for example with toluene, xylene, chlorobenzene or dichloromethane. Toluene is particularly suitable.

If the substituted aniline and glycidol are employed in the reaction in a mixing ratio in which the substituted aniline is reacted virtually completely, the latter only remains in a small residual amount in the resultant 3-phenylaminopropane-1,2-diol. Even if the yield, based on glycidol, then drops again, this is advantageous, since recovery of the substituted aniline is then superfluous.

The invention therefore furthermore relates to an embodiment of the process which is characterised in that the substituted aniline and glycidol are reacted with one another in a molar ratio of approximately 1:1.2. In this case, only from 1 to 10% of the substituted aniline remain unreacted, and can subsequently easily be removed, for example by recrystallisation of the product. With this embodiment of the invention, product yields of up to about 65%, based on glycidol, and from 70 to 75%, based on substituted aniline, are achieved. In contrast to the previous process, isolation of the product is furthermore advantageously totally superfluous, and the reaction mixture can be employed directly without work-up in the next step of the synthetic sequence. This results in a further considerable saving of work, and material and energy costs.

The substituted 3-phenylaminopropane-1,2-diol obtained by reaction of the substituted aniline with glycidol can, for example, be further reacted with dialkyl carbonate to give substituted phenyloxazolidinone (reaction 2). To this end, the substituted 3-phenylaminopropane-1,2-diol is taken up and reacted in the dialkyl carbonate. The invention thus furthermore relates to a process for the preparation of substituted phenyloxazolidinone compounds which is characterised in that (a) firstly glycidol dissolved in the melt of the substituted aniline is converted into substituted 3-phenylaminopropane-1,2-diol, and (b) this is subsequently reacted with dialkyl carbonate to give the substituted phenyloxazolidinone compound.

If the substituted aniline and glycidol are employed in reaction 1, as described above, in a mixing ratio in which the substituted aniline is reacted virtually completely, the resultant reaction mixture comprising the substituted 3-phenylaminopropane-1,2-diol can be employed directly. The invention therefore also relates to a further embodiment of the process which is characterised in that (a) firstly the substituted aniline and glycidol is reacted with one another in a molar ratio of approximately 1:1.2 to give substituted 3-phenylaminopropane-1,2-diol, and (b) this is subsequently reacted, without prior purification, with dialkyl carbonate to give the substituted phenyloxazolidinone compound.

The removal of the impurities from the first step is carried out simply during work-up in the second step. In the case of reaction of aminobenzonitrile, an overall yield of up to 55% (based on glycidol) arises over the two steps, compared with at best 30% over the two steps in the old process.

Dialkyl carbonates which can be employed are, for example, dimethyl carbonate, dieethyl carbonate, dipropyl carbonate, dibutyl carbonate, dipentyl carbonate and dihexyl carbonate. Preference is given to the use of dieethyl carbonate.

The examples explain the invention without being restricted thereto.

### EXAMPLE 1

71.6 g (0.60 mol) of 4-aminobenzonitrile are introduced into a heatable flask fitted with a stirrer and warmed to 60° C. While the temperature is maintained, 24.6 g (0.32 mol) of R-(+)-glycidol) are added dropwise over the course of one hour, and the mixture is stirred for 16 hours and allowed to solidify. The cooled melt is taken up in 200 ml of toluene at 60° C.; the mixture is stirred overnight at room temperature. The crystalline precipitate is filtered off, washed with toluene and dried at 40° C. under reduced pressure.

The yield is 61%, based on aminobenzonitrile, or 77%, based on glycidol.

### EXAMPLE 2

23.9 g (0.20 mol) of 4-aminobenzonitrile are introduced into a heatable flask fitted with a stirrer and warmed to 60° C. While the temperature is maintained, 18.1 g (0.24 mol) of glycidol are added dropwise over the course of one hour, and the mixture is stirred for 6 hours and left to stand overnight. The batch is subsequently dissolved in 80 ml of water of warm water at 60° C., allowed to cool overnight and stirred at 0° C. for 4 hours. The crystalline substance obtained after removal of the solvent is dried overnight at 50° C. under reduced pressure.

The yield is 74%, based on aminobenzonitrile, or 62%, based on glycidol.

### EXAMPLE 3

23.9 g (0.20 mol) of 4-aminobenzonitrile are introduced into a heatable flask fitted with a stirrer and warmed to 60° C. While the temperature is maintained, 18.1 g (0.24 mol) of R-(+)-glycidol) are added dropwise over the course of one hour, and the mixture is stirred for 6 hours and left to stand overnight. 152.7 g of diethyl carbonate and 1.4 g of potassium tertbutoxide (as catalyst) are subsequently added, the temperature is increased to about 80° C., and alcohol liberated by the reaction is distilled off over the course of 1 hour. The remaining solvent is then removed at 60° C. under reduced pressure, the residue which remains is dissolved in 123 g of ethanol at 60° C., the mixture is stirred overnight at room temperature and then for 4 hours at 0° C., and the resultant precipitate is filtered off and dried at 50° C. under reduced pressure.

The yield is 60%, based on aminobenzonitrile, or 50%, based on glycidol.

### EXAMPLE 4 (COMPARATIVE EXAMPLE)

34.7 g (0.46 mol) of R-(+)-glycidol are added dropwise over the course of 5 hours at 75° C. to a mixture of 27.1 g (0.23 mol) of aminobenzonitrile. The solution is cooled to 0° C. and stirred overnight. The deposited crystals are filtered off and dissolved in 140 ml of boiling ethyl acetate. The solution is cooled to 0° C. and stirred overnight. The crystallised-out substance is filtered off, washed with ice-cooled ethyl acetate and dried under reduced pressure. Concentration of the ethyl acetate filtrate to about one third, further stirring overnight at 0°, filtering off, washing and drying under reduced pressure enable a second batch to be obtained. The overall yield of L-4-(2,3-dihydroxypropylamino)benzonitrile is 67%, based on aminobenzonitrile, or 34%, based on glycidol.

1. Process for the preparation of substituted 3-phenylaminopropane-1,2-diols by reaction of an appropriately substituted aniline with glycidol, characterised in that glycidol dissolved in the melt of the substituted aniline is reacted.

2. Improved process according to claim 1, characterised in that the substituted aniline used is aminobenzonitrile.

3. Process according to claim 2, characterised in that the substituted aniline used is 4-aminobenzonitrile.

4. Process according to claim 1, characterised in that glycidol is reacted in the form of one of its optically active enantiomers.

5. Process according to claims 4, characterised in that R-(+)-glycidol is reacted.

6. Process according to claim 1, characterised in that the substituted aniline and glycidol are reacted with one another in a molar ratio of from 1.5 to 2.5:1, preferably in a molar ratio of approximately 2:1.

7. Process according to claim 1, characterised in that unreacted substituted aniline remaining in the resultant reaction product is subsequently removed by treatment with a suitable solvent.

8. Process according to claim 7, characterised in that the solvent used is toluene.

9. Process according to claim 1, characterised in that the substituted aniline and glycidol are reacted with one another in a molar ratio of approximately 1:1.2.

10. Process for the preparation of substituted phenyloxazolidinone compounds, characterised in that
(a) firstly a substituted 3-phenylaminopropane-1,2-diol is prepared by the process according to claim 1, and
(b) this is subsequently reacted with dialkyl carbonate to give the substituted phenyloxazolidinone compound.

11. Process according to claim 10, characterised in that
(a) firstly a substituted 3-phenylaminopropane-1,2-diol is prepared by a process wherein substituted aniline and glycidol are reacted with one another in a molar ratio of approximately 1:1.2, and
(b) this is subsequently reacted, without prior purification, with dialkyl carbonate to give the substituted phenyloxazolidinone compound.

12. Process according to claim 11, characterised in that the dialkyl carbonate employed is diethyl carbonate.
